(19) United States
(12) Patent Application Publication
(10) Pub. No.: US 2022/0330898 A1
(43) Pub. Date: Oct. 20, 2022

(54) INTRACARDIAC DEVICE AND METHODS OF USE

(71) Applicant: ABIOMED, Inc., Danvers, MA (US)

(72) Inventors: Ali Hassan Almedhychy, Danvers, MA (US); Anjan K. Chakrabarti, Danvers, MA (US)

(73) Assignee: ABIOMED, Inc., Danvers, MA (US)

(21) Appl. No.: 17/719,768

(22) Filed: Apr. 13, 2022

Related U.S. Application Data
(60) Provisional application No. 63/176,676, filed on Apr. 19, 2021.

(51) Int. Cl. A61B 5/00; A61M 60/165; A61B 5/346; A61B 5/33

(52) U.S. Cl. CPC A61B 5/7264; A61M 60/165; A61B 5/6869; A61B 5/346; A61B 5/33; A61M 2230/65

(57) ABSTRACT

Improvements to intracardiac devices such as intracardiac blood pump assemblies, and associated methods. In one example, the present technology includes systems and methods for pacing the heart, and/or performing cardiac ablation using electrodes mounted on a portion of the intracardiac device. In another example, the present technology includes systems and methods for detecting mural thrombi in a patient's heart using electrical sensors or ultrasonic phased arrays mounted on the intracardiac device. In another example, the present technology includes systems and methods for detecting tissue changes and reactions in heart tissue during treatment using one or more temperature sensors. In another example, the present technology includes an improved distal tip for use with an intracardiac device. In another example, the present technology includes systems and methods for maintaining an intracardiac device in a desired position within a patient's heart using magnets or ultrasonic phased arrays mounted on the intracardiac device.

### CROSS-REFERENCE TO RELATED APPLICATIONS

The present application claims priority to U.S. Provisional Application No. 63/176,676 filed Apr. 19, 2021, the disclosure of which is incorporated by reference in its entirety.

### BACKGROUND

Intracardiac blood pump assemblies can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

### BRIEF SUMMARY

The present technology relates to improvements to intracardiac devices such as intracardiac blood pump assemblies.

In one aspect, the present technology includes systems and methods for pacing the heart, and/or performing cardiac ablation using electrodes mounted on a portion of the intracardiac device. For example, for an intracardiac device that is configured to be inserted into a patient's right heart, one or more sensors and one or more electrodes may be mounted on the intracardiac device at a point which will come to rest over triangle of Koch. The intracardiac device may be configured to sense an abnormality (e.g., an arrythmia), and pace the heart using the electrodes. Likewise, an intracardiac device may be configured to sense an abnormality, and perform cardiac ablation to shut down the nerve bundles responsible for that abnormality.

In this regard, the disclosure describes an intracardiac blood pump assembly, comprising: an elongate catheter; an impeller housing coupled to a distal end of the elongate catheter, the impeller housing surrounding an impeller configured to draw blood into a cannula coupled to a distal end of the impeller housing; one or more electrical sensors mounted on the cannula and configured to sense electrical pulses within an AV node or a His bundle of a patient's heart when the intracardiac blood pump assembly is inserted into a right ventricle of a patient's heart; and one or more electrical emitters mounted on the cannula and configured to emit a pulse of electrical energy into the AV node or the His bundle when the intracardiac blood pump assembly is inserted into a right ventricle of a patient's heart. In some aspects, the assembly further comprises one or more processors configured to: receive one or more signals of the one or more electrical sensors; and determine a presence of an abnormal heart beat based on the one or more electrical sensors. In some aspects, the one or more processors are further configured to, when the intracardiac blood pump assembly is inserted into a right ventricle of a patient's heart, cause the one or more electrical emitters to emit a pulse of electrical energy into the AV node or the His bundle sufficient to pace the patient's heart. In some aspects, the one or more processors are further configured to, when the intracardiac blood pump assembly is inserted into a right ventricle of a patient's heart, cause the one or more electrical emitters to emit a pulse of electrical energy into the AV node or the His bundle sufficient to disable one or more nerve bundles responsible for the abnormal heart beat.

In another aspect, the present technology includes systems and methods for detecting mural thrombi in a patient's heart using electrical sensors. For example, an intracardiac blood pump assembly may be equipped with two or more electrical sensors capable of emitting and sensing electrical signals, and thus measuring the impedance across of portion of heart tissue. The measured impedance may be used to determine whether the tissue in question is normal or abnormal (e.g., thrombus), so that any mural thrombi can be treated prior to operating the intracardiac blood pump. This determination may be based, for example, on a comparison of the measured impedance value to pre-determined, tissue-characteristic impedance values.

In this regard, the disclosure describes a system for sensing tissue characteristics, comprising: (i) an intracardiac device configured to be inserted into a patient's heart; (ii) one or more electrical emitters mounted on the intracardiac device and configured to emit an input pulse of electrical energy into a first portion of a tissue within the patient's heart; (iii) one or more electrical sensors mounted on the intracardiac device and configured to sense a corresponding pulse of electrical energy at a second portion of the tissue within the patient's heart, the corresponding pulse of electrical energy resulting from the conduction of the input pulse through the tissue; and (iv) one or more processors configured to: compare a voltage of the input pulse with a voltage of the corresponding pulse; determine an impedance value of the tissue based on the comparison; and determine a tissue type of the tissue based at least in part on the impedance value. In some aspects, the one or more electrical emitters comprises a first emitter mounted at a first location on the intracardiac device, and the one or more electrical sensors comprises a first sensor mounted at a second location on the intracardiac device. In some aspects, the first sensor is further configured to emit pulses of electrical energy, and the first emitter is further configured to sense pulses of electrical energy. In some aspects, the one or more processors being configured to determine a tissue type of the tissue based at least in part on the impedance value comprises being configured to compare the impedance value to a reference impedance value. In some aspects, the one or more processors being configured to compare the impedance value to a reference impedance value further comprises being configured to determine whether the impedance value differs from the reference impedance value by a predetermined amount or percentage. In some aspects, the system further comprises a controller configured to cause the one or more electrical emitters to emit the input pulse. In some aspects, the controller is further configured to receive the corresponding pulse from the one or more electrical sensors. In some aspects, the controller comprises the one or more processors. In some aspects, the intracardiac device comprises an intracardiac blood pump.

The disclosure also describes a method for sensing tissue characteristics, comprising: inserting an intracardiac device into a patient's heart, the intracardiac device having one or more electrical emitters and one or more electrical sensors; emitting an input pulse of electrical energy into a first portion of a tissue within the patient's heart using the one or more electrical emitters; sensing a corresponding pulse of electrical energy at a second portion of the tissue within the patient's heart using the one or more electrical sensors, the corresponding pulse of electrical energy resulting from the conduction of the input pulse through the tissue; comparing, using one or more processors of a processing system, a voltage of the input pulse with a voltage of the corresponding pulse; determining, using the one or more processors, an impedance value of the tissue based on the comparison; and determining, using the one or more processors, a tissue type of the tissue based at least in part on the impedance value. In some aspects, the one or more electrical emitters comprises a first emitter mounted at a first location on the intracardiac device, and the one or more electrical sensors comprises a first sensor mounted at a second location on the intracardiac device. In some aspects, the first sensor is further configured to emit pulses of electrical energy, and the first emitter is further configured to sense pulses of electrical energy. In some aspects, determining a tissue type of the tissue based at least in part on the impedance value comprises comparing the impedance value to a reference impedance value. In some aspects, comparing the impedance value to a reference impedance value further comprises determining whether the impedance value differs from the reference impedance value by a predetermined amount or percentage. In some aspects, the reference impedance value is generated by: emitting an input pulse of electrical energy into a first portion of a reference tissue within the patient's heart using the one or more electrical emitters; sensing a corresponding pulse of electrical energy at a second portion of the reference tissue within the patient's heart using the one or more electrical sensors, the corresponding pulse of electrical energy resulting from the conduction of the input pulse through the reference tissue; comparing, using the one or more processors, a voltage of the input pulse with a voltage of the corresponding pulse; and determining, using the one or more processors, the reference impedance value of the tissue based on the comparison. In some aspects, the intracardiac device comprises an intracardiac blood pump.

In another aspect, the present technology includes systems and methods for detecting tissue changes and reactions in heart tissue during treatment using one or more temperature sensors. For example, to monitor whether an intracardiac device is producing an undesirable effect on heart tissue with which it is in contact (e.g., at a distal tip of the intracardiac device), the intracardiac device may be equipped with one or more temperature sensors to monitor temperature changes that may be indicative of such effects.

In this regard, the disclosure describes a system for sensing tissue characteristics, comprising: (i) an intracardiac device configured to be inserted into a patient's heart; (ii) one or more first temperature sensors mounted on the intracardiac device and configured to measure a first temperature of a first portion of a tissue within the patient's heart; and (iii) one or more processors configured to: compare the first temperature with a reference temperature value; and determine whether the first portion of tissue is exhibiting an adverse reaction to the intracardiac device based on the comparison. In some aspects, the system further comprises one or more second temperature sensors configured to measure a second temperature of a second portion of tissue within the patient's heart. In some aspects, the one or more processors being configured to compare the first temperature with a reference temperature value comprises being configured to compare the first temperature with the second temperature. In some aspects, the system further comprises a controller configured to receive the first temperature from the one or more first temperature sensors. In some aspects, the controller comprises the one or more processors. In some aspects, the intracardiac device comprises an intracardiac blood pump.

The disclosure also describes a method for sensing tissue characteristics, comprising: inserting an intracardiac device into a patient's heart, the intracardiac device having one or more first temperature sensors mounted on the intracardiac device; sensing a first temperature of a first portion of a tissue within the patient's heart using the one or more first temperature sensors; comparing, using one or more processors of a processing system, the first temperature with a reference temperature value; and determining, using the one or more processors, whether the first portion of tissue is exhibiting an adverse reaction to the intracardiac device based on the comparison. In some aspects, the method further comprises measuring a second temperature of a second portion of tissue within the patient's heart using one or more second temperature sensors. In some aspects, comparing the first temperature with a reference temperature value comprises comparing the first temperature with the second temperature. In some aspects, the intracardiac device comprises an intracardiac blood pump.

In another aspect, the present technology includes systems and methods for detecting mural thrombi in a patient's heart using intracardiac echocardiography. For example, an intracardiac blood pump assembly may be equipped with a linear phased array or circular phased array near its distal tip to provide high-resolution intracardiac echocardiography to detect mural thrombi so that they can be treated prior to operating the intracardiac blood pump. Likewise, the present technology includes systems and methods for determining and maintaining the position of an intracardiac blood pump within a patient's heart using a linear phased array or circular phased array mounted on the intracardiac blood pump.

In this regard, the disclosure describes an improved intracardiac blood pump assembly, comprising: an intracardiac blood pump configured to be inserted into a patient's heart; and an ultrasonic phased array mounted on the intracardiac blood pump and configured to provide intracardiac echocardiography. In some aspects, the ultrasonic phased array is an ultrasonic linear phased array. In some aspects, the ultrasonic phased array is an ultrasonic circular phased array. In some aspects, the ultrasonic circular phased array is configured to provide a two-dimensional image. In some aspects, the ultrasonic circular phased array is configured to provide a three-dimensional image. In some aspects, the intracardiac blood pump comprises an atraumatic extension at a distal end of the intracardiac blood pump; and the ultrasonic phased array is mounted on the atraumatic extension. In some aspects, the improved intracardiac blood pump assembly further comprises one or more processors configured to determine a presence of mural thrombi based on output of the ultrasonic phased array. In some aspects, the one or more processors being configured to determine a presence of mural thrombi based on output of the ultrasonic phased array comprises being configured to compare the output of the ultrasonic phased array to one or more reference images. In some aspects, the one or more processors being configured to determine a presence of mural thrombi based on output of the ultrasonic phased array comprises being configured to provide the output of the ultrasonic phased array to a neural network trained to identify mural thrombi in medical images. In some aspects, the improved intracardiac blood pump assembly further comprises one or more processors configured to, when the intracardiac blood pump is inserted within the patient's heart, determine a position of the intracardiac blood pump within the patient's heart. In some aspects, the one or more processors being configured to determine a position of the intracardiac blood pump within the patient's heart comprises being configured to compare the output of the ultrasonic phased array to one or more reference images. In some aspects, the one or more processors being configured to determine a position of the intracardiac blood pump within the patient's heart comprises being configured to provide the output of the ultrasonic phased array to a neural network trained to identify anatomical features in medical images.

In another aspect, the present technology includes an improved distal tip for use with an intracardiac device. In that regard, the improved distal tip of the present technology may have a symmetric or asymmetric closed loop shape, and may further be configured with sections of differing stiffness that contribute to bias the tip to anchor itself (and thus the intracardiac device) in a desired location and/or orientation within a patient's heart. The closed loop of this improved distal tip may also reduce the chances of the tip injuring or becoming entangled with cardiac structures. In some aspects of the technology, the improved distal tip may also include or serve as an electrical sensor or emitter (e.g., for use in measuring impedance, detecting arrythmias, pacing, or performing cardiac ablation, as described above and further below), and/or an antenna for sending or receiving signals. In that regard, in some aspects, the looped tip may be composed of a conductive material or include one or more conductive members so that the looped tip itself may function as a sensor, emitter, and/or antenna.

In this regard, the disclosure describes an intracardiac blood pump assembly, comprising: an elongate catheter; an impeller housing coupled to a distal end of the elongate catheter, the impeller housing surrounding an impeller configured to draw blood into a cannula coupled to a distal end of the impeller housing; a distal cage coupled to a distal end of the cannula, configured to allow blood to be drawn into or expelled out of the cannula; and an atraumatic extension coupled to the distal cage, the atraumatic extension comprising a closed loop. In some aspects, the closed loop of the atraumatic extension has a symmetric shape. In some aspects, the closed loop of the atraumatic extension has an asymmetric shape. In some aspects, the closed loop of the atraumatic extension includes a parametric curve. In some aspects, the closed loop of the atraumatic extension includes a Euler curve. In some aspects, the atraumatic extension comprises a proximal section and a distal section, wherein the proximal section is stiffer than the distal section. In some aspects, the atraumatic extension comprises one or more wires or electrically conductive members. In some aspects, the atraumatic extension is further configured to act as an antenna. In some aspects, the atraumatic extension is further configured to act as an electrical sensor. In some aspects, the atraumatic extension is further configured to act as an electrical emitter.

In another aspect, the present technology includes systems and methods for maintaining an intracardiac device in a desired position within a patient's heart using magnets. For example, a permanent magnet such as a rare-earth magnet may be mounted on a portion of the intracardiac device that is intended to anchor against a portion of the heart (e.g., against a ventricle wall), and a second magnet of sufficient strength may be positioned near that portion of the heart (e.g., external to the patient, within the patient's chest but outside the heart, implanted within the heart, etc.) in order to pull that portion of the intracardiac device into the intended anchoring location in the heart and/or hold the intracardiac device in that intended anchoring location.

In this regard, the disclosure describes a system for maintaining position of an intracardiac device, comprising: an intracardiac device configured to be inserted into a patient's heart; a ferromagnetic element mounted on the intracardiac device; and a first magnet configured to be positioned outside of a cavity of the patient's heart, and to attract the ferromagnetic element while the intracardiac device is inserted within the cavity to bias the intracardiac device in a given location within the cavity. In some aspects, the ferromagnetic element is not a magnet. In some aspects, the ferromagnetic element is a magnet. In some aspects, the ferromagnetic element is a rare-earth magnet. In some aspects, the first magnet is a permanent magnet. In some aspects, the first magnet is a rare-earth magnet. In some aspects, the first magnet is an electromagnet. In some aspects, the first magnet is configured to be positioned external to the patient. In some aspects, the first magnet is configured to be positioned within a portion of tissue of the patient's heart. In some aspects, the first magnet is configured to be positioned within a pericardium of the patient's heart. In some aspects, the first magnet is configured to be positioned within an epicardium of the patient's heart.

### BRIEF DESCRIPTION OF DRAWINGS

FIG. 1 depicts an exemplary intracardiac blood pump assembly configured for left heart support, in accordance with aspects of the disclosure;

FIG. 2 depicts an exemplary intracardiac blood pump assembly configured for right heart support, in accordance with aspects of the disclosure;

FIG. 3 is a functional block diagram of an exemplary intracardiac blood assembly in accordance with aspects of the disclosure;

FIG. 4 depicts an intracardiac blood pump assembly inserted into a right ventricle, in accordance with aspects of the disclosure;

FIG. 5 depicts an intracardiac blood pump assembly inserted into a left ventricle which includes a mural thrombus, in accordance with aspects of the disclosure;

FIG. 6A depicts a sectional view of an exemplary configuration of an atraumatic extension at a distal tip of the intracardiac blood pump assembly of FIG. 5, in accordance with aspects of the disclosure;

FIG. 6B is a diagram illustrating exemplary signals sent and received by the electrical emitter and sensor positioned on the distal tip of FIG. 6A, in accordance with aspects of the disclosure;

FIGS. 7A-7E are sectional views of a distal end of an intracardiac blood pump assembly illustrating various exemplary sensor arrangements, in accordance with aspects of the disclosure;

FIG. 8A is a sectional view of a portion of an intracardiac blood pump assembly illustrating one example of how wires from the sensor may exit the proximal end of the cannula in accordance with aspects of the disclosure;

FIG. 8B is a sectional view of a portion of the intracardiac blood pump assembly of FIG. 8A illustrating one example of how wires from the sensor may enter the distal end of the catheter, in accordance with aspects of the disclosure;

FIG. 9A depicts an intracardiac blood pump assembly with an ultrasonic linear phased array mounted near its distal end, in accordance with aspects of the disclosure;

FIG. 9B depicts an intracardiac blood pump assembly with an ultrasonic circular phased array mounted near its distal end, in accordance with aspects of the disclosure;

FIG. 9C depicts an intracardiac blood pump assembly with an ultrasonic circular phased array inserted into a left ventricle, with the phased array providing an ultrasonic scan of the aortic valve, in accordance with aspects of the disclosure;

FIG. 9D depicts an intracardiac blood pump assembly with an ultrasonic circular phased array inserted into a left ventricle, with the phased array providing an ultrasonic scan of the left ventricle, in accordance with aspects of the disclosure;

FIG. 10 depicts an intracardiac blood pump assembly with a looped atraumatic extension inserted into a left ventricle, in accordance with aspects of the disclosure;

FIG. 11 depicts an intracardiac blood pump assembly with an atraumatic extension on which a magnet is mounted, in accordance with aspects of the disclosure;

FIG. 12 is a flow diagram of an exemplary method for determining tissue type, in accordance with aspects of the disclosure; and

FIG. 13 is a flow diagram of an exemplary method for determining the existence of an adverse reaction to an intracardiac device, in accordance with aspects of the disclosure.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, methods, and devices described herein, certain illustrative examples will be described. Although various examples may describe intracardiac blood pump assemblies, it will be understood that the improvements of the present technology may also be adapted and applied to other types of medical devices such as electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices.

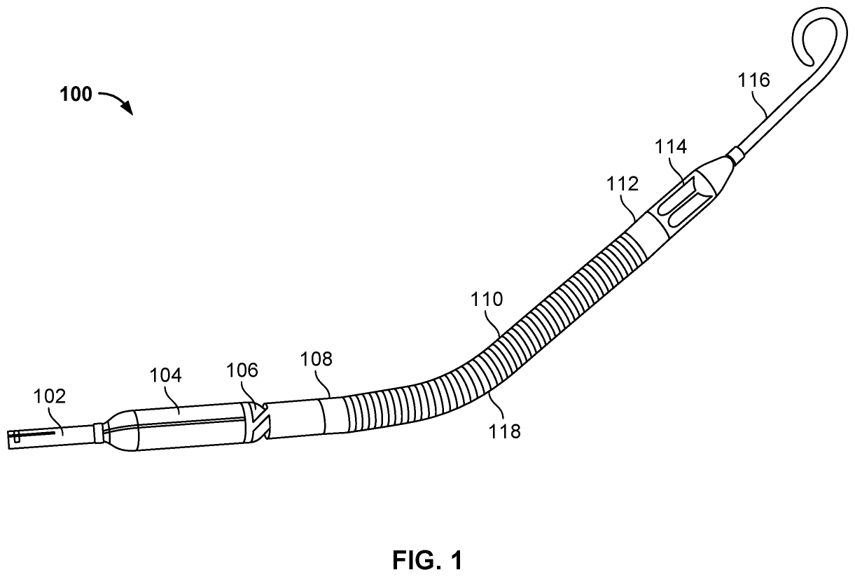

FIG. 1 depicts an exemplary intracardiac blood pump assembly 100 adapted for left heart support. In that regard, the intracardiac blood pump assembly 100 includes an elongate catheter 102, a motor 104, a cannula 110, a blood inflow cage 114 arranged at or near the distal end 112 of the cannula 110, a blood outflow cage 106 arranged at or near the proximal end 108 of the cannula 110, and an optional atraumatic extension 116 arranged at the distal end of the blood inflow cage 114.

Motor 104 is configured to rotatable drive an impeller (not shown), thereby generating suction sufficient to draw blood into cannula 110 through the blood inflow cage 114, and to expel the blood out of cannula 110 through the blood outflow cage 106. In that regard, the impeller may be positioned distal of the blood outflow cage 106, for example, within the proximal end 108 of the cannula 110 or within a housing coupled to the proximal end 108 of the cannula 110. In some aspects of the technology, rather than the impeller being driven by an in-dwelling motor 104, the impeller may instead be coupled to an elongate drive shaft (or drive cable) which is driven by a motor located external to the patient.

Catheter 102 may house electrical lines coupling the motor 104 to one or more electrical controllers or other sensors. Alternatively, where the impeller is driven by an external motor, an elongate drive shaft may pass through catheter 102. Catheter 102 may also serve as a conduit for wires connecting the electrical sensors described further below to one or more controllers, power sources, etc. located outside the patient's body. Catheter 102 may also include a purge fluid conduit, a lumen configured to receive a guidewire, etc.

The blood inflow cage 114 includes one or more apertures or openings configured to allow blood to be drawn into cannula 110 when the motor 104 is operating. Likewise, blood outflow cage 106 includes one or more apertures or openings configured to allow blood to flow from the cannula 110 out of the intracardiac blood pump assembly 100. Blood inflow cage 114 and outflow cage 106 may be composed of any suitable bio-compatible material(s). For example, blood inflow cage 114 and/or blood outflow cage 106 may be formed out of bio-compatible metals such as stainless steel, titanium, or biocompatible polymers such as polyurethane. In addition, the surfaces of blood inflow cage 114 and/or blood outflow cage 106 may be treated in various ways, including, but not limited to etching, texturing, or coating or plating with another material. For example, the surfaces of blood inflow cage 114 and/or blood outflow cage 106 may be laser textured.

Cannula 110 may include a flexible hose portion. For example, cannula 110 may be composed, at least in part, of a polyurethane material. In addition, cannula 110 may include a shape-memory material. For example, cannula 110 may comprise a combination of a polyurethane material and one or more strands or coils of a shape-memory material such as Nitinol. Cannula 110 may be formed such that it includes one or more bends or curves in its relaxed state, or it may be configured to be straight in its relaxed state. In that regard, in the exemplary arrangement shown in FIG. 1, the cannula 110 has a single pre-formed anatomical bend 118 based on the portion of the left heart in which it is intended to operate. Despite this bend 118, the cannula 110 may nevertheless also be flexible, and may thus be capable of straightening (e.g., during insertion over a guidewire), or bending further (e.g., in a patient whose anatomy has tighter dimensions). Further in that regard, cannula 110 may include a shape-memory material configured to allow the cannula 110 to be a different shape (e.g., straight or mostly straight) at room temperatures, and to form bend 118 once the shape-memory material is exposed to the heat of a patient's body.

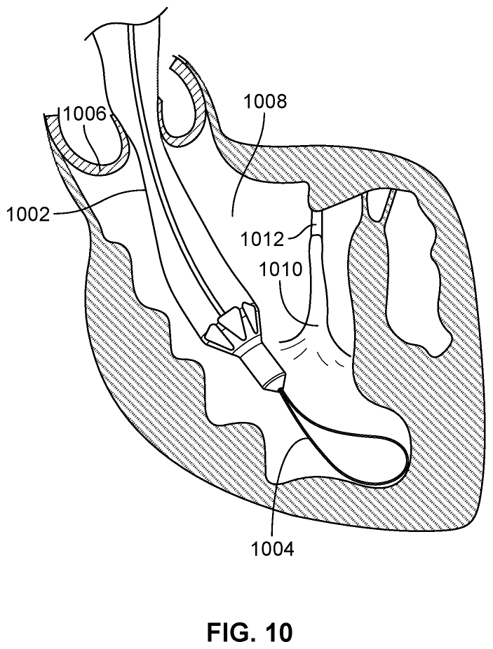

Atraumatic extension 116 assists with stabilizing and positioning the intracardiac blood pump assembly 100 in the correct position in the patient's heart. Atraumatic extension 116 may be solid or tubular. If tubular, atraumatic extension 116 may be configured to allow a guidewire to be passed through it to further assist in the positioning of the intracardiac blood pump assembly 100. Atraumatic extension 116 may be any suitable size. For example, atraumatic extension 116 may have an outer diameter in the range of 4-8 Fr. Atraumatic extension 116 may be composed, at least in part, of a flexible material, and may be any suitable shape or configuration such as a straight configuration, a partially curved configuration, a pigtail-shaped configuration as shown in the example of FIG. 1, or a looped configuration, as described further below with respect to FIG. 10. Atraumatic extension 116 may also have sections with different stiffnesses. For example, atraumatic extension 116 may include a proximal section that is stiff enough to prevent it from buckling, thereby keeping the blood inflow cage 114 in the desired location, and a distal section that is softer and has a lower stiffness, thereby providing an atraumatic tip for contact with a wall of the patient's heart and to allow for guidewire loading. In such a case, the proximal and distal sections of the atraumatic extension 116 may be composed of different materials, or may be composed of the same material, treated to provide different stiffnesses.

Notwithstanding the foregoing, as mentioned above, atraumatic extension 116 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

Intracardiac blood pump assembly 100 may be inserted percutaneously. For example, when used for left heart support, intracardiac blood pump assembly 100 may be inserted via a catheterization procedure through the femoral artery or axillary artery, into the aorta, across the aortic valve, and into the left ventricle. Once positioned in this way, the intracardiac blood pump assembly 100 delivers blood from the blood inflow cage 114, which sits inside the left ventricle, through cannula 110, to the blood outflow cage 106, which sits inside the ascending aorta. As will be explained further below, in some aspects of the technology, intracardiac blood pump assembly 100 may be configured such that bend 118 will rest against a predetermined portion of the patient's heart when the intracardiac blood pump assembly 100 is in a desired location. Likewise, the atraumatic extension 116 may be configured such that it rests against a different predetermined portion of the patient's heart when the intracardiac blood pump assembly 100 is in the desired location.

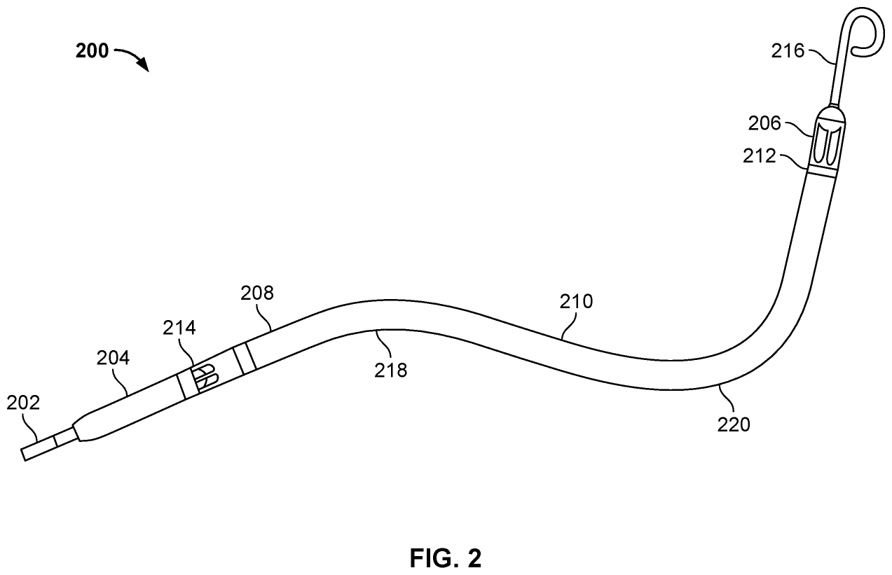

FIG. 2 depicts an exemplary intracardiac blood pump assembly 200 adapted for right heart support. In that regard, the intracardiac blood pump assembly 200 includes an elongate catheter 202, a motor 204, a cannula 210, a blood inflow cage 214 arranged at or near the proximal end 208 of the cannula 210, a blood outflow cage 206 arranged at or near the distal end 212 of the cannula 210, and an optional atraumatic extension 216 arranged at the distal end of the blood outflow cage 206.

As with the exemplary assembly of FIG. 1, motor 204 is configured to rotatable drive an impeller (not shown), thereby generating suction sufficient to draw blood into cannula 210 through the blood inflow cage 214, and to expel the blood out of cannula 210 through the blood outflow cage 206. In that regard, the impeller may be positioned distal of the blood inflow cage 214, for example, within the proximal end 208 of the cannula 210 or within a housing coupled to the proximal end 208 of the cannula 210. Here as well, in some aspects of the technology, rather than the impeller being driven by an in-dwelling motor 204, the impeller may instead be coupled to an elongate drive shaft (or drive cable) which is driven by a motor located external to the patient.

The cannula 210 of FIG. 2 serves the same purpose, and may have the same properties and features described above with respect to cannula 110 of FIG. 1. However, in the exemplary arrangement shown in FIG. 2, the cannula 210 has two pre-formed anatomical bends 218 and 220 based on the portion of the right heart in which it is intended to operate. Here again, despite the existence of bends 218 and 220, the cannula 210 may nevertheless also be flexible, and may thus be capable of straightening (e.g., during insertion over a guidewire), or bending further (e.g., in a patient whose anatomy has tighter dimensions). Further in that regard, cannula 210 may include a shape-memory material configured to allow the cannula 210 to be a different shape (e.g., straight or mostly straight) at room temperatures, and to form bends 218 and/or 220 once the shape-memory material is exposed to the heat of a patient's body.

The catheter 202 and atraumatic extension 216 of FIG. 2 serve the same purpose and may have the same properties and features described above with respect to catheter 102 and atraumatic extension 116 of FIG. 1. Likewise, other than being located at opposite ends of the cannula from those of FIG. 1, the blood inflow cage 214 and blood outflow cage 206 of FIG. 2 are similar to the blood inflow cage 114 and blood outflow cage 106 of FIG. 1, and thus may have the same properties and features described above.

Like the exemplary assembly of FIG. 1, the intracardiac blood pump assembly 200 of FIG. 2 may also be inserted percutaneously. For example, when used for right heart support, intracardiac blood pump assembly 200 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 200 delivers blood from the blood inflow cage 214, which sits inside the inferior vena cava, through cannula 210, to the blood outflow cage 206, which sits inside the pulmonary artery.

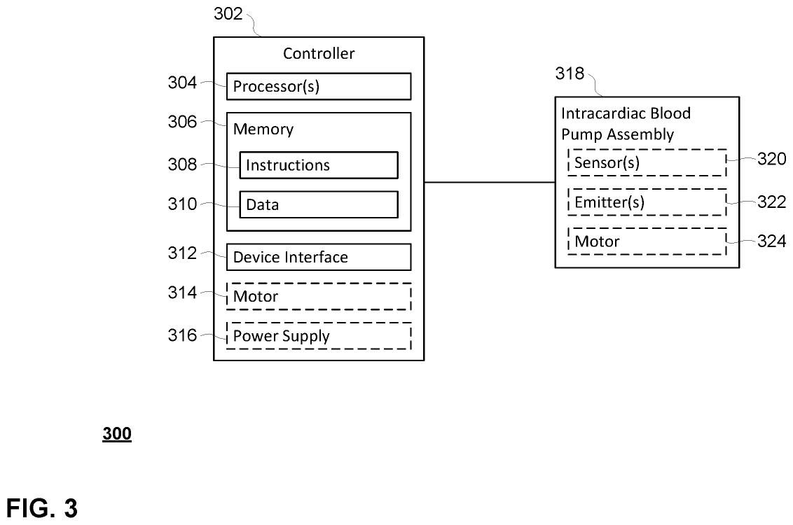

FIG. 3 is a functional block diagram of an exemplary system in accordance with aspects of the disclosure. In that regard, in the example of FIG. 3, the system 300 comprises an intracardiac blood pump assembly 318 and a controller 302. The intracardiac blood pump assembly 318 may take any form, including those shown in the exemplary blood pump assemblies 100 and 200 of FIG. 1 or 2, respectively. In addition, the intracardiac blood pump assembly 318 of FIG. 3 may optionally include one or more sensors 320 (e.g., electrical sensors, temperature sensors, ultrasonic linear or circular phased arrays, etc.), one or more emitters 322 (e.g., electrical emitters, RF antennas, ultrasonic linear or circular phased arrays, etc.), and a motor 324 configured to rotatably drive an impeller (e.g., in instances where the motor is configured to be inserted into the patient). Notwithstanding the foregoing, the present technology may also be employed in systems comprising an intracardiac device other than a blood pump assembly.

In the example of FIG. 3, the controller 302 includes or more processors 304 coupled to memory 306 storing instructions 308 and data 310, and an interface 312 with the intracardiac blood pump assembly 318. Controller 302 may additionally include an optional motor 314 (e.g., in instances where the impeller is driven by a motor located external to the patient via an elongate drive shaft) and/or a power supply 316 (e.g., to power an in-dwelling motor 324, sensors 320, emitters 322, etc.). The interface 312 with intracardiac blood pump assembly 318 may be any suitable interface. In that regard, interface 312 may be configured to enable one one-way or two-way communication between the controller 302 and the intracardiac blood pump assembly 318. Interface 312 may further be configured to provide power to one or more sensors 320 or emitters 322 (e.g., those described in the various figures below), and/or to an in-dwelling motor 324.

Controller 302 may take any form. In that regard, controller 302 may comprise a single modular unit, or its components may be distributed between two or more physical units. Controller 302 may further include any other components normally used in connection with a computing device such as a user interface. In that regard, controller 302 may have a user interface that includes one or more user inputs (e.g., buttons, touchscreen, keypad, keyboard, mouse, microphone, etc.); one or more electronic displays (e.g., a monitor having a screen or any other electrical device that is operable to display information, one or more lights, etc.); one or more speakers, chimes or other audio output devices; and/or one or more other output devices such as vibrating, pulsing, or haptic elements.

The one or more processors 304 and memory 306 described herein may be implemented on any type of computing device(s), including customized hardware or any type of general computing device. Memory 306 may be of any non-transitory type capable of storing information accessible by the processor(s) 304, such as a hard-drive, memory card, optical disk, solid-state, tape memory, or similar structure.

Instructions 308 may include programming configured to receive readings from the one or more sensors 320 and control the signals to be produced by the one or more emitters 322.

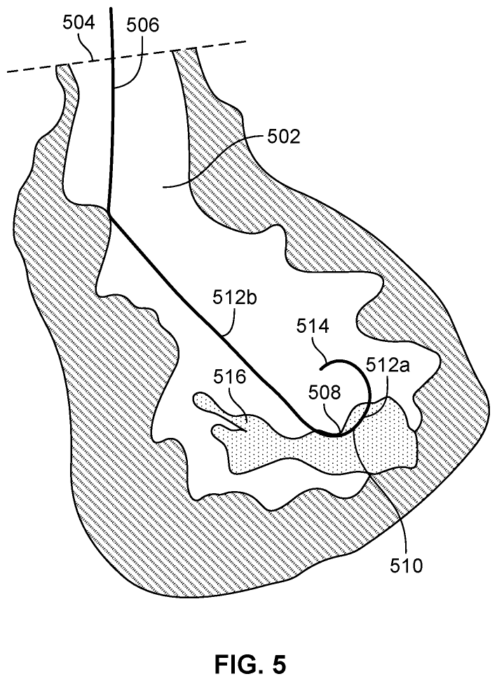

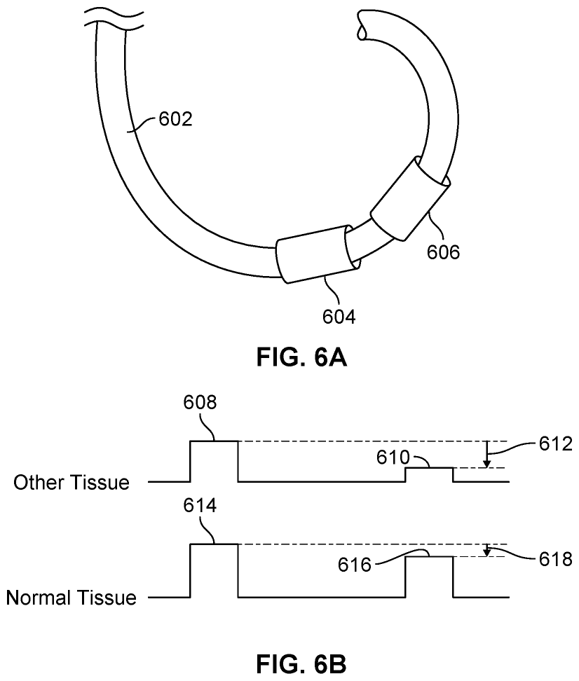

Data 310 may include data for calibrating and/or interpreting the signals of the one or more sensors 320 and emitters 322, as well as data regarding impedance characteristics of representative heart tissue (e.g., as described below with respect to FIGS. 5 and 6), temperature characteristics of representative heart tissue (e.g., as described below with respect to FIG. 5), and other relevant criteria. Controller 302 may further be configured to store past readings from sensors 320 in memory 306, e.g., for use in making the determinations described below.

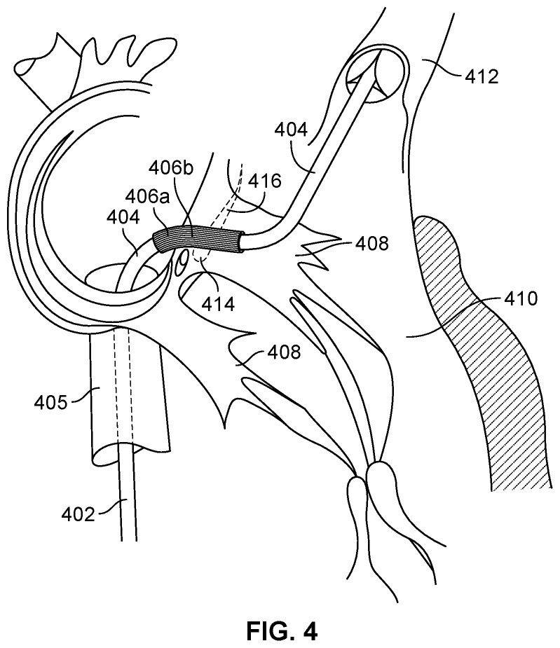

FIG. 4 depicts a cross-sectional view of a right ventricle, with an exemplary intracardiac blood pump assembly 402 inserted therein, in accordance with aspects of the disclosure. More specifically, FIG. 4 shows intracardiac blood pump assembly 402 inserted through the inferior vena cava 405, across the paraseptal leaflets 408 of the tricuspid valve, into the right ventricle 410, and into the pulmonary artery 412. In the example of FIG. 4, the intracardiac blood pump assembly 402 includes one or more electrical sensors 406a and one or more electrical emitters 406b located at or near a bend in the cannula 404. In the orientation shown in FIG. 4, the one or more electrical sensors 406a are positioned over the triangle of Koch such that the signals of the AV node 414 and/or the His bundle 416 can be sensed, and abnormalities (e.g., arrythmias such as atrial fibrillation, high or low ST segment readings indicating potential subendocardial or transmural ischemia, etc.) may be identified. Likewise, with the one or more electrical emitters 406b positioned over the AV node 414 and/or the His bundle 416, cardiac ablation may be performed to disable the nerve bundles responsible for an arrythmia, and/or pacing may be performed to correct an abnormal heart rhythm.

FIG. 5 depicts a cross-sectional view of a left ventricle 502 which includes within it a mural thrombus 516. FIG. 5 also depicts an exemplary intracardiac blood pump assembly 506 inserted through the aortic valve 504 into the left ventricle 502 such that its atraumatic extension 514 is in contact with the mural thrombus 516.

In one aspect of the technology, the intracardiac blood pump assembly 506 may be configured with one or more electrical emitters 508 configured to emit a pulse of electrical energy, and one or more electrical sensors 510 configured to measure electrical potential. In such a case, the distal tip of the intracardiac blood pump assembly 506 may be placed in contact with tissue to be tested (e.g., mural thrombus 516), and a controller (e.g., controller 302) may be configured to cause the one or more electrical emitters 508 to provide a pulse of a predetermined amount of electrical energy into the tissue and to measure how much of that electrical energy is sensed by the one or more electrical sensors 510. The controller may be further configured to compare the amount of the electrical energy emitted by the one or more electrical emitters 508 to the amount of electrical energy sensed by the one or more electrical sensors 510 to obtain an impedance value of the tissue in question, and may be further configured to make a determination as to the nature of the tissue (e.g., whether the tissue is normal heart tissue, or abnormal tissue such as a mural thrombus) based on that comparison. This determination may be based, for example, on a comparison of the measured (or calculated) impedance value to pre-determined, tissue-characteristic impedance values (e.g., values based on empirical data regarding average impedance values for normal heart tissue, for thrombi, etc.).

In one aspect of the technology, the intracardiac blood pump assembly 506 may be configured with one or more temperature sensors 512a. In such a case, the distal tip of the intracardiac blood pump assembly 506 may be placed in contact with tissue to be tested, and a controller (e.g., controller 302) may be configured to compare the temperature of the tissue in question provided by the one or more temperature sensors 512a to a reference value, and to make a determination as to the nature of the tissue based on that comparison. For example, an elevated temperature relative to the reference value may indicate that the tissue in question is exhibiting a reaction to being in contact with the intracardiac blood pump assembly 506. In some aspects, the reference value may be a stored temperature reading taken previously by the one or more temperature sensors 512a, e.g., when the distal tip was initially placed in contact with healthy tissue, a history of some or all of the prior temperature readings taken by the one or more temperature sensors 512a, or some value (e.g., average, minimum, maximum) based on that history of prior temperature readings. Likewise, in some aspects, the reference value may be based on one or more temperature readings from one or more temperature sensors 512b mounted on a different portion of the intracardiac blood pump assembly 506. In some aspects, the reference value may be an assumed value based on empirical data regarding average temperatures for normal heart tissue.

Although the example of FIG. 5 shows an intracardiac blood pump assembly 506 which includes both electrical emitters and sensors, as well as temperature sensors, it will be understood that an intracardiac blood pump assembly in accordance with the present technology may include only electrical emitters and sensors. Likewise, an intracardiac blood pump assembly in accordance with the present technology may include only one or more temperature sensors. Further in that regard, although the example of FIG. 5 shows temperature sensors mounted at two different positions, an intracardiac blood pump assembly in accordance with the present technology may include temperature sensors mounted only at a single location.

Further, it should be noted that the exact positions of the one or more electrical emitters 508, the one or more electrical sensors 510, and the one or more temperature sensors 512a and 512b are merely illustrative. Any or all of these may be mounted at different positions and/or structures of the intracardiac blood pump assembly 506.

FIG. 6A depicts a sectional view of an exemplary configuration of an atraumatic extension 602 at a distal tip of the intracardiac blood pump assembly of FIG. 5, in accordance with aspects of the technology. In the example of FIG. 6A, the atraumatic extension includes one or more electrical emitters 604 and one or more electrical sensors 606, which are spaced apart by some predetermined distance. In some aspects of the technology, the structures identified as 604 and 606 may be capable of both emitting and sensing electrical energy, so that measurements may be taken in either direction.

FIG. 6B is a diagram illustrating exemplary signals sent and received by the one or more electrical emitters and the one or more electrical sensors positioned on the distal tip of FIG. 6A. In that regard, the top graph shows an exemplary signal 608 being emitted by the one or more electrical emitters 604 into abnormal tissue (e.g., a mural thrombus), and an exemplary signal 610 being sensed by the one or more electrical sensors 606. This results in a drop in potential indicated by arrow 612, which may be used to determine an impedance value for the tissue in question. In contrast, the bottom graph shows an exemplary signal 614 being emitted by the one or more electrical emitters 604 into normal cardiac tissue, and an exemplary signal 616 being sensed by the one or more electrical sensors 606. As can be seen, this results in a drop in potential indicated by arrow 618 which is different than the drop indicated by arrow 612. In some aspects of the technology, the voltage drop in normal tissue (e.g., that indicated by arrow 618) may be used to determine a reference impedance value for normal cardiac tissue. This reference value may be stored and used by the controller (e.g., controller 302) in subsequent readings to determine whether other tested tissue is normal or abnormal. For example, in some aspects, if a subsequent reading results in an impedance drop that is greater than the stored reference value by some predetermined percentage (e.g., 3%, 5%, 10%, 30%, 50%, etc.) or predetermined amount (e.g., 50 milliohms, 100 milliohms, 1 ohm, etc.), it may be determined that the tissue is abnormal.

Although the example of FIGS. 5, 6A, and 6B have assumed that an impedance value would be calculated, and that a determination of whether the tissue in question is normal or abnormal would be based on the impedance value, in some aspects of the technology, the determination of whether the tissue in question is normal or abnormal may instead be based directly a measured voltage drop. Likewise, although the example of FIG. 6B assumes that the abnormal tissue will have a larger voltage drop (as shown by arrow 612) and thus a higher impedance value than that of the normal tissue (as shown by arrow 618), it will be understood that in some cases, abnormal tissue of interest may be characterized by higher conductivity, and thus a lower impedance value than that of normal tissue.

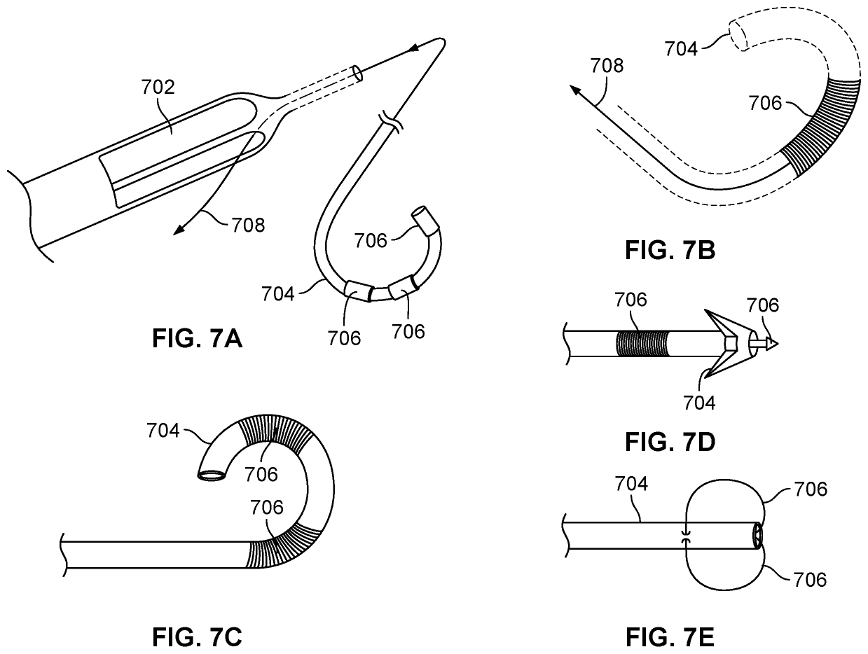

FIGS. 7A-7E are sectional views of a distal end of an intracardiac blood pump assembly illustrating various exemplary sensor arrangements, in accordance with aspects of the disclosure. These exemplary sensor arrangements may be used with any of the intracardiac blood pump assemblies described herein, including those of FIGS. 1-6, and 9.

In that regard, FIG. 7A depicts an intracardiac blood pump assembly having a pigtail-shaped atraumatic extension 704 with three sensors 706. In each of the examples of FIGS. 7A-7E, the described sensors 706 may be electrical sensors and/or electrical emitters, antennas, temperature sensors, or linear or circular phased arrays, as described further above and below. As shown in FIG. 7A, one or more wires 708 configured to carry a signal to and/or from the three sensors 706 extend down the inside of the atraumatic extension, into the distal end of cage 702 (e.g., a blood inflow or blood outflow cage), and out of one of the apertures of cage 702.

FIG. 7B depicts an intracardiac blood pump assembly having a pigtail-shaped atraumatic extension 704 with sensor 706. Here again, one or more wires 708 configured to carry a signal to and/or from the sensor 706 extend down the inside of the atraumatic extension.

FIG. 7C depicts an intracardiac blood pump assembly having a pigtail-shaped atraumatic extension 704 with two sensors 706. Wires may be present to carry a signal to and/or from the sensors 706 as discussed in the preceding examples, but are not depicted in this particular sectional view.

FIG. 7D depicts an intracardiac blood pump assembly having an arrow-head extension 704. Arrow-head extension 704 is configured to anchor the distal tip of the intracardiac blood pump assembly in the cardiac tissue to prevent pump migration, and may be substituted for any of the atraumatic extensions described herein. The intracardiac blood pump assembly of FIG. 7D has two sensors 706, one on the shaft of the extension, and one on the distal tip. Such an arrangement may be useful, for example, where it is desirable to obtain comparative measurements both inside and outside of selected portion of tissue. Wires may be present to carry a signal to and/or from the electrical sensor 706 as discussed in the preceding examples, but are not depicted in this particular sectional view.

FIG. 7E depicts an intracardiac blood pump assembly having a straight atraumatic extension 704 with sensors 706 comprising looped wires. Here as well, signals between the sensors 706 may be carried to and from a controller (e.g., controller 302) by one or more wires that extend down the inside of the atraumatic extension as discussed in the preceding examples, but are not depicted in this particular sectional view.

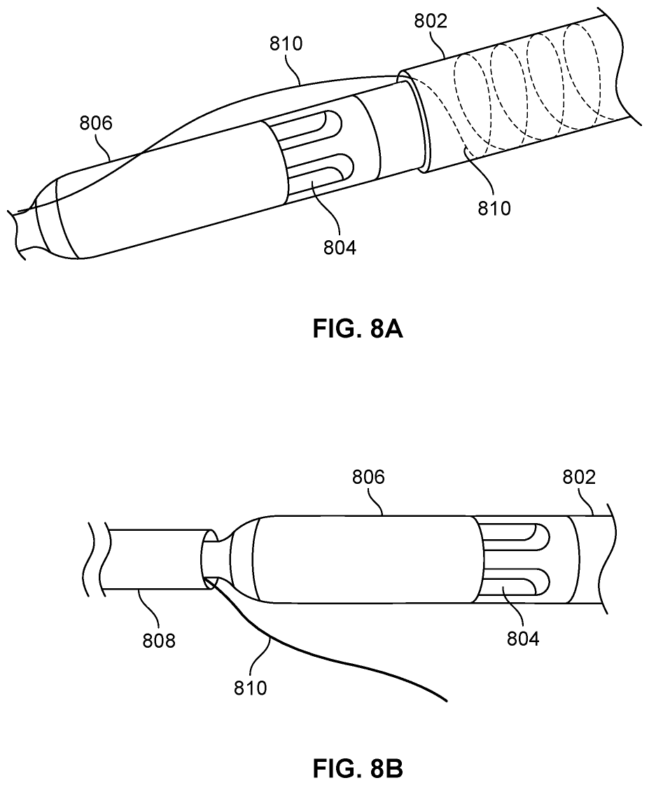

FIG. 8A is a sectional view of a portion of an intracardiac blood pump assembly illustrating one example of how wires from the one or more sensors may exit the proximal end of the cannula in accordance with aspects of the disclosure. In that regard, one or more wires 810 spiral around the cannula 802. The one or more wires 810 may spiral along an inner or outer surface of cannula 802, or may be embedded within the wall of cannula 802 (e.g., molded within the wall of cannula 802). The one or more wires 810 exit cannula 802 where the proximal end of cannula 802 meets up with cage 804 (e.g., a blood inflow or blood outflow cage). In that regard, the one or more wires 810 may exit cannula 802 by protruding out where cannula 802 overlaps with cage 804, or by passing through an aperture of cannula 802. The one or more wires 810 pass over motor 806 and continue in the proximal direction.

FIG. 8B is a sectional view of a portion of the blood pump assembly of FIG. 8A, illustrating one example of how the one or more wires 810 from the sensor may enter the distal end of the catheter 808. In that regard, all numerals shared between FIGS. 8A and 8B denote the same structures. As can be seen, in the example of FIG. 8B, the one or more wires 810 enter into catheter 808 where it overlaps with the proximal end of the housing of motor 806. In some aspects of the technology, the one or more wires 810 may run within a lumen of elongate catheter 808 out of the patient, where they will interface with a controller (e.g., controller 302 and device interface 312).

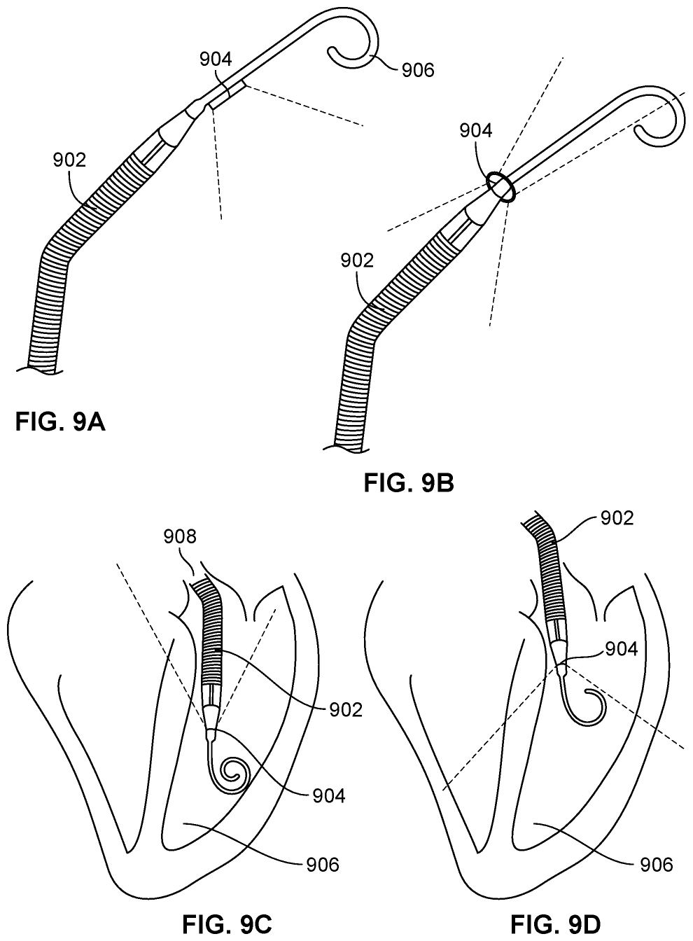

FIG. 9A depicts an intracardiac blood pump assembly 902 with an ultrasonic linear phased array 904 mounted near its distal end, in accordance with aspects of the disclosure. In the example of FIG. 9A, the ultrasonic linear phased array 904 is mounted on a portion of the atraumatic extension 906. However, the ultrasonic linear phased array 904 may be mounted on any suitable portion of the intracardiac blood pump assembly 902. The ultrasonic linear phased array 904 will produce a linear ultrasonic beam, as shown by the dashed lines emanating from element 904. As such, an intracardiac blood pump assembly such as the one shown in FIG. 9A will need to be aimed at the portion of the heart for which an ultrasonic image is desired.

FIG. 9B depicts an intracardiac blood pump assembly 902 with an ultrasonic circular phased array 904, in accordance with aspects of the disclosure. Here as well, although the ultrasonic circular phased array 904 is mounted near the distal end of intracardiac blood pump assembly 902, it may be mounted on any suitable portion of the intracardiac blood pump assembly 902. The ultrasonic circular phased array 904 will produce a conical ultrasonic beam, and may be focused so as to provide a conical sweep in different directions relative to its center, as shown by the dashed lines emanating both proximally and distally from element 904. This may produce a two-dimensional cross-sectional image of what is swept, or a three-dimensional (cross-sectional and axial) image of what is swept. As such, an intracardiac blood pump assembly such as the one shown in FIG. 9B will be able to provide views both in front of and behind the mounting point of the ultrasonic circular phased array 904. In that regard, FIG. 9C shows how an intracardiac blood pump assembly 902 with an ultrasonic circular phased array 904 may be used within a left ventricle 906 to provide a backward-looking ultrasonic scan of the aortic valve 908. Likewise, FIG. 9D shows how an intracardiac blood pump assembly 902 with an ultrasonic circular phased array 904 may be used within a left ventricle 906 to provide a forward-looking ultrasonic scan of the left ventricle 906.

In each of the examples of FIGS. 9A-9D, the present technology may be used to provide an intracardiac echocardiograph within a patient's heart after the intracardiac blood pump assembly 902 has been inserted therein. This provides improved imaging over traditional imaging methods used during insertion of an intracardiac blood pump assembly 902 such as fluoroscopy, which is 2-dimensional and can be inadequate to detect 3-dimensional structures, or transaortic echocardiography, which can be difficult to perform due to poor acoustic windows and/or interference from the intracardiac blood pump assembly 902 (after it has been inserted). Likewise, as dedicated intracardiac echo catheters require their own vascular access, they may not be feasible for use in procedures in which an intracardiac blood pump assembly 902 is being inserted into a patient's heart as well. In contrast, mounting a phased array on the intracardiac blood pump assembly 902 allows for high-resolution imaging to be taken of the portion of the heart into which the intracardiac blood pump assembly 902 has been inserted. This may be advantageous, for example, to ensure that no mural thrombi are present which may become dislodged as a result of running the pump to provide cardiac assistance. In some aspects of the technology, a determination of the presence of mural thrombi may be made by one or more processors of a processing system (e.g., controller 302) based on the output of the ultrasonic phased array. In some aspects of the technology, this determination may be further based on past images of the patient's heart, images taken from other patients, neural networks trained to identify or detect the presence of mural thrombi in medical images, etc.

The ability to take high-resolution images of the portion of the heart into which the intracardiac blood pump assembly 902 has been inserted may also be advantageous for confirming that the intracardiac blood pump assembly 902 has been inserted into a desired location. Likewise, the ability to continue taking high-resolution images from within the heart may allow for the position of the intracardiac blood pump assembly 902 to be periodically rechecked to ensure that it does not shift within the heart over time. In some aspects of the technology, a determination of the position of the intracardiac device within the patient's heart may be made by one or more processors of a processing system (e.g., controller 302) based on the output of the ultrasonic phased array. In some aspects of the technology, this determination may be further based on past images of the patient's heart, images taken from other patients, neural networks trained to identify anatomical features from medical images, etc.

FIG. 10 depicts an intracardiac blood pump assembly 1002 with a looped atraumatic extension 1004, in accordance with aspects of the disclosure. In the example of FIG. 10, the intracardiac blood pump assembly 1002 has been inserted through a patient's aortic valve 1006 into the left ventricle 1008, and has come to rest in a position in which the looped atraumatic extension 1004 is anchored against a wall of the left ventricle 1008.

In some aspects, the looped atraumatic extension 1004 may have a symmetric or asymmetric shape configured to anchor the intracardiac blood pump assembly 1002 in a desired location and/or orientation within the heart and to bias the intracardiac blood pump assembly 1002 against moving out of that desired position. Any suitable curvature may be used for the looped atraumatic extension 1004. For example, in some aspects, the curvature of the looped atraumatic extension 1004 may be based on the anatomical features of a representative heart. In some aspects, the curvature of the looped atraumatic extension 1004 may be based on one or more mathematically derived curves such as a parametric curve, one or more sections of a Euler curve, etc. Likewise, in some aspects, the shape and size of the closed-loop structure of the looped atraumatic extension 1004 may be configured to prevent it from becoming entangled on structures within the heart. For example, the size and shape of the looped atraumatic extension 1004 may be configured to avoid it from catching on valves or other delicate structures such as chordae tendineaa 1010 and papillary muscles 1012. In this way, the looped atraumatic extension 1004 may provide advantages over atraumatic extensions of other shapes (e.g., straight, pigtail, etc.).

In some aspects, the stiffness of the looped atraumatic extension 1004 may also be configured to contribute to its tendency to hold the intracardiac blood pump assembly 1002 in position. For example, the looped atraumatic extension 1004 may have a proximal section which is stiffer than a distal section, allowing the tip to bend where it contacts a wall of the heart to avoid puncturing and/or damaging the tissue, while still allowing the remainder of the looped atraumatic extension 1004 to be stiff enough to resist buckling and maintain the position of the intracardiac blood pump assembly 1002.

The closed loop of the looped atraumatic extension 1004 may be formed from one or more wires or conductive members, or may include one or more wires or conductive members. In that regard, in some aspects, the looped atraumatic extension 1004 may be configured to act as a sensor, emitter, and/or antenna. For example, the looped atraumatic extension 1004 may be configured to emit electrical energy (e.g., to test tissue condition, pace the heart, or perform cardiac ablation), sense electrical energy (e.g., to sense signals propagating through the heart, or to measure electrical pulses emitted by an emitter), and/or to act as an antenna for transmitting or receiving signals (e.g., from a controller such as controller 302).

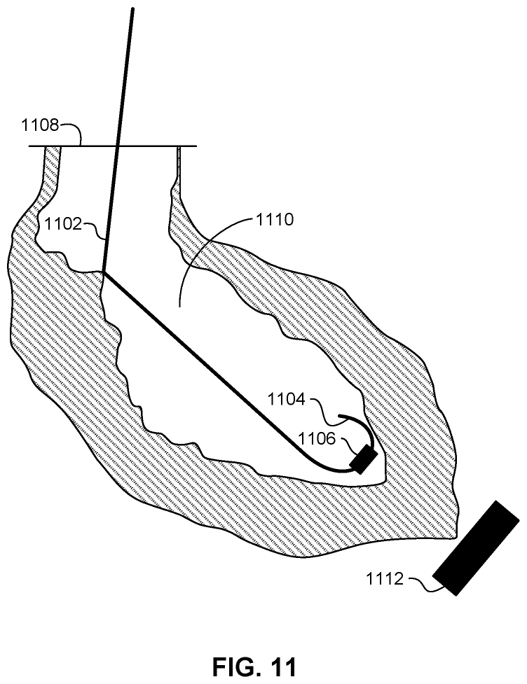

FIG. 11 depicts an intracardiac blood pump assembly 1102 with an atraumatic extension 1104 on which a ferromagnetic element 1106 is mounted, in accordance with aspects of the disclosure. In the example of FIG. 11, the intracardiac blood pump assembly 1102 has been inserted through a patient's aortic valve 1108 into the left ventricle 1110, and has come to rest in a position in which the atraumatic extension 1104 is anchored against a wall of the left ventricle 1110. In order to maintain the intracardiac blood pump assembly 1102 in this position, a magnet 1112 configured to attract ferromagnetic element 1106 is positioned outside the left ventricle 1100. The magnet 1112 may be positioned external to the patient (e.g., on the patient's chest), within the patient's chest cavity but outside the heart, or within tissue of the heart (e.g., in the pericardium, epicardium, etc.). In some aspects of the technology, magnet 1112 and ferromagnetic element 1106 may both be magnets, and may be any type suitable for holding intracardiac blood pump assembly 1102 in this position, including permanent rare-earth magnets. In some aspects of the technology, ferromagnetic element 1106 may be a permanent magnet, and magnet 1112 may be an electromagnet. Further, in some aspects of the technology, ferromagnetic element 1106 may not be magnetic itself, but may instead comprise a material (e.g., iron) which is attracted to magnet 1112.

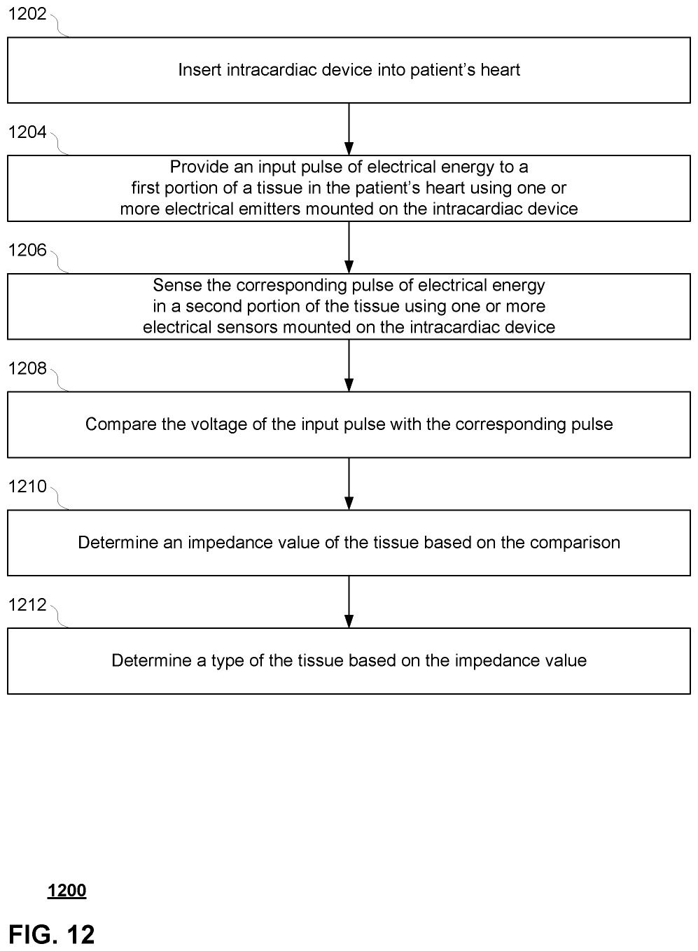

FIG. 12 is a flow diagram of an exemplary method 1200 for determining the type of tissue in which an intracardiac device is in contact, in accordance with aspects of the disclosure. In that regard, in step 1202, an intracardiac device is inserted into a patient's heart. This may involve any suitable intracardiac device, such as those described above with respect to FIGS. 1-11, and may be done in any suitable way, such as percutaneously via any of the catheterization procedures described above with respect to FIGS. 1 and 2.

In step 1204, a pulse of electrical energy is provided to a first portion of tissue in the patient's heart using one or more electrical emitters (e.g., emitter 508, 604) mounted on the intracardiac device. In this example, the one or more electrical emitters produce a pulse of a predetermined time and power, such as shown in signals 608 and 614 of FIG. 6B. The pulse may be initiated and controlled, for example, by a controller of the intracardiac device such as controller 302 of FIG. 3.

In step 1206, a corresponding pulse of electrical energy is sensed at a second portion of the tissue using one or more electrical sensors (e.g., sensor 510, 606) mounted on the intracardiac device. The second portion of tissue may be a portion of tissue displaced by any suitable distance from the first portion of tissue. The corresponding pulse of electrical energy is what results from the conduction of the input pulse through the tissue. As such, the voltage of the corresponding pulse (e.g., signals 610, 616) will differ from the input pulse by some amount based on the conductive characteristics of the tissue, as explained above with respect to FIG. 6B.

In step 1208, the voltage of the input pulse is compared to the voltage of the corresponding pulse received from the electrical sensor. In step 1210, an impedance value is determined based on the comparison of 1208. One or both of steps 1208 and 1210 may be performed, for example, by one or more processors of the controller, e.g., processors 304 of controller 302 of FIG. 3.

In step 1212, the tissue's type (e.g., muscle tissue, mural thrombi, etc.) is determined based on the impedance value, as described above with respect to FIGS. 5, 6A, and 6B. This determination may be made, for example, by one or more processors of the controller, e.g., processors 304 of controller 302 of FIG. 3. In that regard, the determination of tissue type may be based on information in addition to the impedance value determined in step 1210. For example, the impedance value in step 1210 may be compared to a reference impedance value, such as an impedance value measured on other tissue within the patient's heart, empirical data regarding the average impedance of healthy cardiac tissue, etc. Likewise, the determination of tissue type may be based in part on whether the determined impedance value differs from a reference impedance value by some predetermined percentage (e.g., 3%, 5%, 10%, 30%, 50%, etc.) or predetermined amount (e.g., 50 milliohms, 100 milliohms, 1 ohm, etc.).

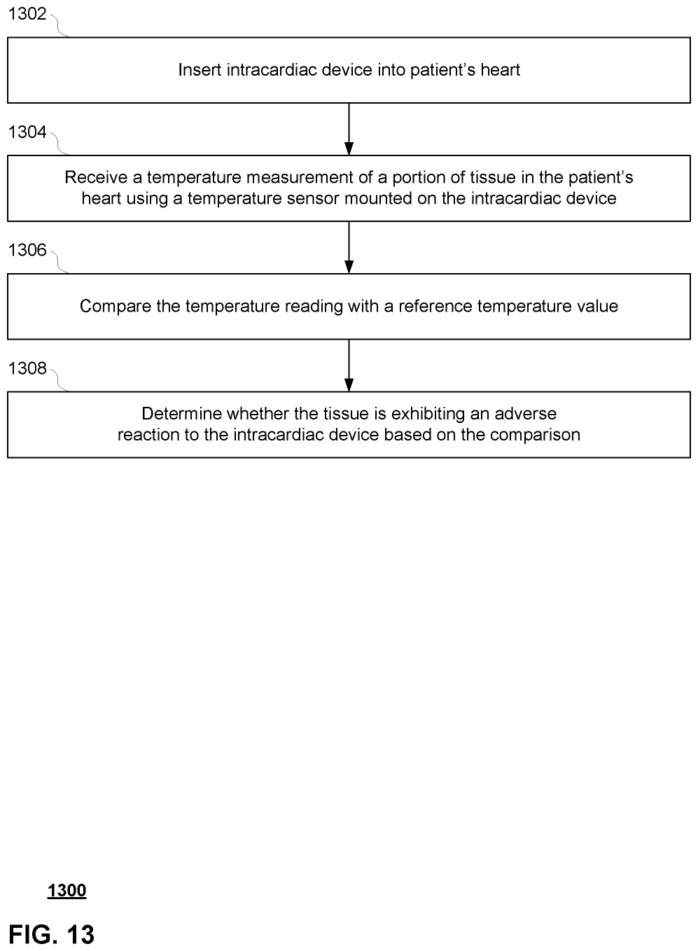

FIG. 13 is a flow diagram of an exemplary method 1300 for determining the existence of an adverse reaction to an intracardiac device, in accordance with aspects of the disclosure. In that regard, in step 1202, an intracardiac device is inserted into a patient's heart. Here again, this may involve any suitable intracardiac device, such as those described above with respect to FIGS. 1-11, and may be done in any suitable way, such as percutaneously via any of the catheterization procedures described above with respect to FIGS. 1 and 2.

In step 1304, a temperature measurement of a portion of tissue in the patient's heart is received from a temperature sensor (e.g., temperature sensor 512a) mounted on the intracardiac device, e.g., by a controller of the intracardiac device such as controller 302 of FIG. 3.

In step 1306, the temperature measurement is compared to a reference temperature value. As described above with respect to FIG. 5, this reference temperature value may be a stored temperature reading taken previously, e.g., when the temperature sensor was initially placed in contact with healthy tissue, a history of some or all of the prior temperature readings taken by the temperature sensor, or some value (e.g., average, minimum, maximum) based on that history of prior temperature readings. Likewise, in some aspects, the reference value may be based on one or more temperature readings from one or more other temperature sensors mounted on a different portion of the intracardiac blood pump assembly. In some aspects, the reference value may be an assumed value based on empirical data regarding average temperatures for normal heart tissue.

In step 1308, a determination is made as to whether the tissue is exhibiting an adverse reaction to the intracardiac device based on the comparison of step 1306, as described above with respect to FIG. 5. For example, an elevated temperature relative to the reference value may indicate that the tissue in question is swelling as a result of being in contact with the intracardiac blood pump assembly 506. This determination may be made, for example, by one or more processors of the controller, e.g., processors 304 of controller 302 of FIG. 3. Here as well, this determination may be based on information in addition to the comparison of step 1306.

From the foregoing and with reference to the various figures, those skilled in the art will appreciate that certain modifications can also be made to the present disclosure without departing from the scope of the same. While several aspects of the disclosure have been shown in the figures, it is not intended that the disclosure be limited thereto, as it is intended that the disclosure be as broad in scope as the art will allow and that the specification be read likewise. Therefore, the above description should not be construed as limiting, but merely as exemplifications of particular aspects of the present technology.

1. A system for sensing tissue characteristics, comprising:
an intracardiac device configured to be inserted into a patient's heart;
one or more electrical emitters mounted on the intracardiac device and configured to emit an input pulse of electrical energy into a first portion of a tissue within the patient's heart;
one or more electrical sensors mounted on the intracardiac device and configured to sense a corresponding pulse of electrical energy at a second portion of the tissue within the patient's heart, the corresponding pulse of electrical energy resulting from the conduction of the input pulse through the tissue; and
one or more processors configured to:
compare a voltage of the input pulse with a voltage of the corresponding pulse;
determine an impedance value of the tissue based on the comparison; and
determine a tissue type of the tissue based at least in part on the impedance value.

2. The system of claim 1, wherein the one or more electrical emitters comprises a first emitter mounted at a first location on the intracardiac device, and the one or more electrical sensors comprises a first sensor mounted at a second location on the intracardiac device.

3. The system of claim 2, wherein the first sensor is further configured to emit pulses of electrical energy, and the first emitter is further configured to sense pulses of electrical energy.

4. The system of claim 1, wherein the one or more processors being configured to determine a tissue type of the tissue based at least in part on the impedance value comprises being configured to compare the impedance value to a reference impedance value.

5. The system of claim 4, wherein the one or more processors being configured to compare the impedance value to a reference impedance value further comprises being configured to determine whether the impedance value differs from the reference impedance value by a predetermined amount or percentage.

6. The system of claim 1, further comprising:
a controller configured to cause the one or more electrical emitters to emit the input pulse.

7. The system of claim 1, wherein the controller is further configured to receive the corresponding pulse from the one or more electrical sensors.

8. The system of claim 7, wherein the controller comprises the one or more processors.

9. The system of claim 1, wherein the intracardiac device comprises an intracardiac blood pump.

10. A method for sensing tissue characteristics, comprising:
inserting an intracardiac device into a patient's heart, the intracardiac device having one or more electrical emitters and one or more electrical sensors;
emitting an input pulse of electrical energy into a first portion of a tissue within the patient's heart using the one or more electrical emitters;
sensing a corresponding pulse of electrical energy at a second portion of the tissue within the patient's heart using the one or more electrical sensors, the corresponding pulse of electrical energy resulting from the conduction of the input pulse through the tissue;
comparing, using one or more processors of a processing system, a voltage of the input pulse with a voltage of the corresponding pulse;
determining, using the one or more processors, an impedance value of the tissue based on the comparison; and
determining, using the one or more processors, a tissue type of the tissue based at least in part on the impedance value.

11. The method of claim 10, wherein the one or more electrical emitters comprises a first emitter mounted at a first location on the intracardiac device, and the one or more electrical sensors comprises a first sensor mounted at a second location on the intracardiac device.

12. The method of claim 11, wherein the first sensor is further configured to emit pulses of electrical energy, and the first emitter is further configured to sense pulses of electrical energy.

13. The method of claim 10, wherein determining a tissue type of the tissue based at least in part on the impedance value comprises comparing the impedance value to a reference impedance value.

14. The method of claim 13, wherein comparing the impedance value to a reference impedance value further comprises determining whether the impedance value differs from the reference impedance value by a predetermined amount or percentage.

15. The method of claim 13, wherein the reference impedance value is generated by:
emitting an input pulse of electrical energy into a first portion of a reference tissue within the patient's heart using the one or more electrical emitters;
sensing a corresponding pulse of electrical energy at a second portion of the reference tissue within the patient's heart using the one or more electrical sensors, the corresponding pulse of electrical energy resulting from the conduction of the input pulse through the reference tissue;
comparing, using the one or more processors, a voltage of the input pulse with a voltage of the corresponding pulse; and
determining, using the one or more processors, the reference impedance value of the tissue based on the comparison.

16. The method of claim 10, wherein the intracardiac device comprises an intracardiac blood pump.

17-63. (canceled)
